(19) United States
(12) Patent Application Publication
(10) Pub. No.: US 2015/0343232 A1
(43) Pub. Date: Dec. 3, 2015

(54) METHOD OF COMBINED TREATMENT OF MALIGANT TUMORS

(71) Applicants: Sergey PLETNEV, (US); Vladimir PEFTIEV, (US); Andrei PLETNEV, (US)

(72) Inventors: Sergey PLETNEV, MINSK (BY); Andrei PLETNEV, MINSK (BY); Vladimir PEFTIEV, MINSK (BY)

(21) Appl. No.: 14/654,588

(22) PCT Filed: Dec. 19, 2013
(86) PCT No.: PCT/IB2013/061137

§ 371 Date: Jun. 22, 2015

(30) Foreign Application Priority Data
Dec. 20, 2012 (EA) 201300496

(51) Int. Cl. A61N 2/00; A61N 2/02; A61M 37/00; A61N 7/00

(52) U.S. Cl. CPC A61N 2/002; A61N 7/00; A61N 2/004; A61N 2/02; A61M 37/0092; A61M 2037/0007

(57) ABSTRACT

The invention relates to oncology.

The object of this invention is to provide reliable malignant tumor treatment methods, to reduce toxicity of the method and to enhance the role of immunity during the rehabilitation period.

The above objects are to be achieved as follows.

A method for combined treatment of malignant tumors comprises administering a sensitizing agent and exposing a malignant tumor to wave irradiation, wherein ultrasonic radiation is used as a wave irradiation.

This invention relates to the field of oncology.

International standards and guidelines for treating various malignant tumors have been developed and published nowadays.

The following principal malignant tumor therapy methods are known:

operative therapy;

radiotherapy;

chemotherapy;

immunotherapy.

The aggressive operative approach is the major method of treatment of localized tumors. However, such an approach requires extremely wide excision of surrounding tissues. The problem resides in the fact that the thicker a tumor is, the greater degree of lodgement of tumor cells, therefore, large areas of a “visible healthy” tissue around the tumor need to be removed. It is officially established that a distance from a tumor edge to the resection margin should not be less than 3 cm. [Minimum clinical recommendations of the European Society for Medical Oncology (ESMO)-Moscow-Medicine-2004].

Radiation therapy is also carried out at the locally advanced stages and is characterized by the same disadvantages as the operative therapy. Furthermore, it results in the damage of surrounding tissues and total radiation exposure.

The same disadvantages are characteristic of the systemic chemotherapy. To date, advantages of the immunotherapy have not been reliably proven yet.

Recently, the combination therapy methods have proved to be much more effective. One of such methods is the dynamic phototherapy method disclosed in RU Patent No. 2466759. According to this method, 2 or 3 hours prior to treatment, a patient is intravenously injected a photosensitizer, a substance, which when irradiated with light, activates a drug that afterwards disintegrates tumor cells.

One of such drugs is Chlorine e⁶. Chlorine-based drugs are characterized by expressed photodynamic activity: in some cases, the coefficient of inhibition of growth of a number of tumors grafted to rats reached 89.8%.

Chlorine e⁶ relates to non-toxic compounds, thereby allowing a dose of the administered photosensitizer to be reduced and, hence, a toxicity thereof.

To increase stability and efficiency of the chlorine-based drugs, a composition was developed (BY Patent No. 5651, titled “A Drug for Photodynamic Therapy of Malignant Tumors”) based on porphyrins, and namely on Photolon-chlorine e⁶ 40-90 and polyvinylpyrrolidone 10-60.

Comparative studies of chlorine e⁶ and Photolon prepared according to the procedure described in Examples 1-3 have demonstrated that Photolon has a higher specific activity compared to that of chlorine e⁶ in relation to tumors grafted to rats (sarcoma M-1 and alveolar liver cancer PC-1).

The closest known method to the combined photodynamic therapy of the alveolar liver cancer PC-1 grafted in subcutaneous tissue of a rat comprises carrying out anesthetic care, administering a photosensitizer into the tumor and irradiating the tumor with laser (BY patentNo. 12177C1 of Aug. 30 2009).

According to the above, a 5% Photolon ointment is applied to the tumor by using phonophoresis in a continuous mode at the ultrasonic frequency of 880 kGz and the intensity of 0.7 W/cm² for 10 minutes. In two ours the remaining ointment is washed off and the tumor is laser-irradiated.

In this case, the phonophoresis of the Photolon ointment allows to improve photosensitizer penetration and leads to an intensive selective accumulation of the drug in the tumor.

Furthermore, irradiating of the photosensitizer accumulated in the tumor tissue with light of a respective wavelength activates it to induce a photochemical reaction resulting in the formation of singlet oxygen that exhibits high oxidizing capacity, destroying thereby tumor cells (necrosis, apoptosis).

Application and phonophoresis followed by the photodynamic therapy result in the maximum percentage of necrosis area in the group in 2 hours-42.57±16.19 (selected case-78.5).

The present invention provides that the ultrasonic exposure exhibits modifying properties resulting in the reduced time required to reach efficient concentration of photosensitizer in the tumor tissue and enhances the antitumor effect of the photodynamic therapy in general.

The results, however, are extremely uneven in the group (scatter-±16.19%) and, as yet, are far from being stable.

The object of this invention is to provide a reliable method of treatment of a malignant tumor.

Another object of this invention is to reduce toxicity of the method.

An additional object of this invention is to enhance the role of immunity during the rehabilitation period.

The above objects are to be achieved as described herein under.

The method of combined treatment of malignant tumor comprises: administering a sensitizing agent and/or a chemical therapeutic agent, saturating a tumor or a malignant tumor with this agent and exposing the tumor to wave irradiation. The method is characterized by that the ultrasonic radiation is used as a wave action factor. Despite the fact that the ultrasonic radiation is also used in the closest prior art solution (the prototype), it cannot produce a sufficient therapeutic effect since the ultrasonic radiation is used at the early stage of the agent administration as an phonophoretic agent and the sensitizing agent is not activated in the full tumor volume. Using ultrasonic radiation at a saturation stage increases the therapy efficiency and it should be noted that photodynamic irradiation is not required. which also expands possibilities of the method to embrace the cases in which optical irradiation is counter-indicative. It is preferable to use ultrasonic radiation of higher frequency for this technology, for example, with a frequency of 1.3 MHz. To reduce heating effects, pulsed ultrasonic radiation is used.

Further improvement of the method provides additional use of the low-frequency pulsed magnetic field (LFPMF). The pulsed magnetic field increases the efficiency of activation of an acting agent and, as experiments demonstrated, produces an additional direct effect on the tumor cells.

The treatment is preferably administered in 10-15-minute sessions.

A further improvement of this invention consists in that an immunomobilization treatment is given to a patient by using LFPMF. It should be noted that the same equipment is used for this course and the rehabilitation effect is accelerated which is proved by blood tests.

The immobilization treatment is preferably administered in 10-15-minute sessions daily, with a number of sessions being 5-10 up to obtaining a change of blood parameters.

Experiments were carried out in the N.N. Alexandrov Republican Research and Practical Center of Oncology and Medical Radiology using 200 white outbred mail and female rats with a grafted M-1 sarcoma tumor.

The study procedures were conducted subject to regulatory documents. Photolon as the most accessible option was used as a sensitizing agent. In principle, other sensitizing agents or chemotherapeutic drugs may be used.

The antitumor efficiency was assessed by staining the necrotic cells with a blue dye solution in rats. After sacrificing the rats, the tumor was dissected and fixed in the formalin solution. Then sections were made across the largest tumor cross section. The sections were recorded with a digital camera and computer-processed. The necrosis area percentage was determined by the ratio of the stained necrosis area to the area of a respective histotopographic section.

The following groups of rats were used in the experiment by types of treatment:

TABLE 1

| | | | Number |
|---|---|---|---|
| No. | Type of treatment | Necrosis area | of sections |
| 1. | Control group | 3.2 ± 0.02 | 10 |
| 2. | Photolon (intravenously) | 9.43 ± 1.05 | 30 |
| 3. | Photolon + ultrasound | 78.3 ± 2.23 | 30 |
| 4. | Photolon + LFMF + US + LFMF | 80.6 ± 2.24 | 35 |
| 5. | Ultrasound | 62.5 ± 2.08 | 30 |
| 6. | LFMF | 63.8 ± 2.82 | 30 |

The control group and the group with intravenously injected Photolon were not exposed to any external treatment.

The group exposed to a combined effect of Photolon and ultrasound demonstrated good results when exposed to 1.3 MHz ultrasound with a pulse repetition frequency of 50 Hz. In addition, the rats were exposed to ultrasound after a specific interval elapsed from Photolon injection.

The best result (80.6 ±2.24) was obtained by combined effect of LFMF and ultrasonic radiation 3 hours after Photolon injection followed by exposure to 100 Hz LFMF (4 days, 10 minutes each). An additional LFMF effect also improved blood parameters, increased hemoglobin and reduced leukocytes.

It should be also noted that reproducibility of results in groups was high compared to the prototype.

The antitumor effectiveness of specific actions was additionally assessed. High effectiveness helps to significantly expand the possibilities of the combined therapy to treat deep tumors as well.

What we claim is:

1. A method for combined treatment of malignant tumors comprising administering a sensitizing agent and exposing a malignant tumor to wave irradiation, characterized in that the ultrasonic radiation is used as a wave treatment.

2. The method according to claim 1, characterized in the additional use of the low-frequency pulsed magnetic field as a wave treatment.

3. The method according to claims 1 and 2, characterized in that the treatment is administered in 10-15-minute sessions.

4. The method according to claim 3, characterized in that the treatment course is followed by an immunomobilization course by means of exposure to low-frequency pulsed magnetic field (LFPMF).

5. The method according to claim 4, characterized in that immobilization treatment is administered in 10-15-minute sessions daily, with a number of sessions being 5-10 up obtaining a change of blood parameters.
